(19) United States
(12) Patent Application Publication
(10) Pub. No.: US 2002/0182189 A1
(43) Pub. Date: Dec. 5, 2002

(54) Compositions and methods for the repair and construction of bone and other tissue

(76) Inventor: Chia Ning (Sophia) Chang, Taipei (TW)

Correspondence Address: Joseph A. Mahoney, MAYER, BROWN & PLATT, P.O. Box 2828, Chicago, IL, 60690-2828, US

(21) Appl. No.: 09/837,217

(22) Filed: Apr. 19, 2001

(51) Int. Cl. A61K 48/00; C12N 5/08; C12N 15/861

(52) U.S. Cl. 424/93.21; 435/372; 435/456

(57) ABSTRACT

The invention relates to novel compositions comprising genetically engineered cells and one or more polymers. In an additional aspect, the present invention relates to a method for repairing tissue, for example, cranioskeletal or maxillary bone defects, comprising tranducing the BMP-2 gene into bone marrow stromal cells which are harvested from a subject, combining the genetically engineered cells with at least one polymer, and implanting the combination at the site of the defect. The BMP-2 protein is advantageously produced as long as the tranduced gene stays in the cells.

### FIELD OF THE INVENTION

The present invention relates to compositions and methods for repair and construction of bone, cartilage, muscle, adipose and fibrous tissues, and the enhancement of healing of such tissues.

### DESCRIPTION OF THE RELATED ART

Over one million procedures in the United States each year involve bone and cartilage replacement (Langer, R., et al., 1993, Tissue Engineering, SCIENCE. 920:260-266.1). The current supply of bone marrow does not meet the demand for bone grafts Sequences of various bone morphogenic proteins (BMP) are known and have been successfully cloned. Since the genes of the BMP family were successfully cloned, large quantities of individual BMP proteins have been made available by recombinant-DNA technology in hope of assisting bone formation. For example, Yasko et al. first demonstrated the effects of recombinant human BMP-2 on bone formation in the rat segmental femoral-defect model (Yasko, A. W., et al., The Healing of Segmented Defects, Induced by Recombinant Human Bone Morphogenetic Protein (ehBMP-2): a Radiographic, Histological, and Biomechanical Study in Rats, J. BONE JOINT SURG. 74:659-670 (1992)). Studies of this nature have been performed in the sheep femur (Gerhart, T. N., et al., Healing Segmented Femoral Defects in Sheep Using Recombinant Human Bone Morphogenetic Protein, CLIN. ORTHOP. 293:317-326 (1993)), and in canine spine and mandible models. Although the results are encouraging in studies in which BMP protein is implanted directly to a defective area, many disadvantages continue to exist. For example, a large amount of protein (up to milligram quantities) is often required to stimulate significant new bone formation in vivo. This requirement for large quantities of the protein increases the risk of unwanted side effects. Moreover, the lack of a system to deliver proteins in a continuous manner over time may further hamper this technique. (Cook, S. D., et al., In vivo Evaluation of Recombinant Human Osteogenic Protein Implants as a Bone Graft Substitute for Spinal Fusions, SPINE 19:1655-1663 (1994).

Additionally, autologous or autogenous vascularized bone grafts may be harvested from temporal, scapular, radial, rib, iliac, fibular, metatarsal bones, but they have the following shortcomings: (1) Grafts are limited in supply; (2) the bone regenerated from peptides, (rhBMPs) demineralized bone powder, or combinations of both are small in size (Zegzula, H. D., et al., 1997, Bone Formation with Use of rhBMP-2 (Recombinant Human Bone Morphogenetic Protecin (rhBMP-2), J. BONE JOINT SURG. AM. 79(12):1778-1790; Niederwanger, M., et al., 1996, Demineralized Bone Matrix Supplied by Bone Banks for a Carrier of Recombinant Human Bone Morphogenetic Protein (rhBMP-2: A Substitute for Autogenic Bone Grafts J. ORAL IMPLANTOL, 22(3-4):210-215; and (3) allografts are limited in usage because of immunological rejections, transmission of infectious diseases, premature resorption, and donor shortage. (Zegzula, H. D., et al., 1997; Rabies A. B., Chay, S. H., et al., 2000, Healing of Autogenous Intramembranpous Bone in the Presence and Absence of Homologous Demineralized Intramembranous Bone, AM. J. ORTHOD. DENTOFACIAL. ORTHO. p. 117(3):288-297; Niederwanger, M., et al., 1996; Furukawa, T., et al., 2000, Histomorphometric Study on High-Strength Hydroxyapatite/poly (L-lactide) Composite Rods for Internal Fixation of Bone Fractures, J. BIOMED. MATER. RES. 5;50(3):410-419). Consequently, although BMP-2 has been known for 35 years, it is still difficult to use BMP to repair large size defects due to these and other disadvantages.

One approach to repair large defects is to combine polymers with BMP. In recent years, tissue engineering has had some success in employing biocompatible, biodegradable polymers seeded with living cells to create functional tissue. (Langer, R., et al., 1993, Tissue Engineering. SCIENCE. 920; 260-266). The field of tissue engineering has been developing to overcome the problem of donor-tissue scarcity of either whole organs or reconstructive grafts. Earlier work with osteoblasts and PGA/PLA fiber meshes showed that osteoblasts-PGA/PLA constructs can be fabricated and, further, that when they are implanted into an animal host, new bone composition is generated with the final morphology established by the shape of the polymer scaffold.

It is believed that osteoblasts are derived from pluripotential mesenchymal stem cells and may develop into a lineage capable of producing bone, muscle, cartilage, adipose and fibrous tissue (Metcalf, D. 1989, The Molecular Control of Cell Division, Differentiation Commitment and Maturation in Haemopoietc Cells, NATURE 339:27-30); Wiles, M. V., et al., 1991; Multiple Hematopietic Lineages Develop From Embryonic Stem (ES) Cells in Culure, DEVELOPMENT 111:259-267). The ultimate fate of an undifferentiated stem cell is determined by local growth factors and by the environment to which the mesenchymal stem cell is exposed (Goshima, J., Goldberg V. M., et al., 1991, The Origin of Bone Formed in Composite Grafts of Porous Calcium Phosphate Ceramic Loaded With Marrow Cells, CLIN. ORTHOP. 269:274-283).

Bone morphogenic protein (BMP) has been shown to encourage the development of osteoblasts from mesenchymal stem cells. (Lou, J., et al., 1999, Gene Therapy: Adenovirus-Mediated Human Bone Morphogenetic Protein-2 Gene Transfer Induces Mesenchymal Progenitor Cell Proliferation and Differentiation In Vitro and Bone Formation In Vitro. J. ORTHO. RES. 17:43-50).

It was previously demonstrated that BMP-2 gene transfer into mesenchymal progenitor cells could induce undifferentiated stem cells in vitro to follow an osseous lineage resulting in bone formation, although continued incubation for up to 30 days and several medium changes were required. (Lou, J., et al., 1999, Gene Therapy: Adenovirus-Mediated Human Bone Morphogenetic Protein-2 Gene Transfer Induces Mesenchymal Progenitor Cell Proliferation and Differentiation In Vitro and Bone Formation In Vitro, J. ORTHO. RES. 17:43-50). However, it was heretofore unknown as to whether the application of such gene therapy in vivo as disclosed and claimed herein would indeed be effective in reparing bone and other tissues.

### SUMMARY OF THE INVENTION

The in vivo experiments disclosed herein demonstrate that bone and other tissue repair is induced and enhanced by adenovirus BMP-2 transduction. One aspect of the invention relates to novel compositions comprising genetically engineered cells and one or more polymers. In an additional aspect, the present invention relates to a method for repairing tissue, for example, cranioskeletal or maxillary bone defects, comprising tranducing the BMP-2 gene into bone marrow stromal cells which are harvested from a subject, combining the genetically engineered cells with at least one polymer, and implanting the combination at the site of the defect. The BMP-2 protein is advantageously produced as long as the tranduced gene stays in the cells.

### BRIEF DESCRIPTION OF THE DRAWINGS

FIG. 1 is a schematic depicting a method of the present invention.

FIG. 2 is a series of photographs as follows. 2A (Left upper): The maxilla bone in minipigs was exposed by removal of periosteum. 2B (Right upper): The maxilla bony defect in minipigs: 3×1.2 cm² bilaterally. 2C (Left lower): One L-shape resorbable plate was wired on the defect edge to secure the space from soft tissue invagination. 2C (Right lower): The defect was filled by the gene engineered bone marrow mensenchymal stromal cells.

FIG. 3 is a series of photographs of the maxilla after 3 months implantation of the composition of the invention in vivo. Left: control group depicting lack of bone formation. Right: experimental group depicting bone formation and bridging of the defect.

FIG. 4 is a 3D CT image of maxilla repair, 3 months in vivo (Left: control, Right: experiment).

FIG. 5 are photomicrographs of H&E and Von Kossa stainings. A. The H&E staining of the control site showed no bony formation (40×). B. The H&E staining of the experimental site showed cancellous bone formation (40×). C. The Von Kossa staining of the BMP-2 site showed lamilar bone formation (40×). D. The higher power field of the BMP-2 shows bone formation.

FIG. 6 is a Western blot of BMP-2 expression in MSCs.

FIG. 7 is a series of photographs of the cranial defect in minipigs: 2×5 cm². Left upper: the bony defect bilaterally. Right upper: the underlying dura were removed. Lower: the Alloderm was used for dura repair.

FIG. 8 is a series of photographs depicting the cranium in Group I after 3 months implantation in vivo. A. View from cranium. B. Transverse view.

FIG. 9 is a photograph depicting the cranium in Group IV after 6 weeks (A). 3 months (B). implantation in vivo. Transverse view 3 months (C).

FIG. 10 is a 3D CT image of Group I at 3 months (Left: BMP-2 site, Right: βGal site).

FIG. 11 is a 3D CT images of Group II at 3 months (Left: BMP-2 site, Right: βGal site).

FIG. 12 is a 3D CT image of Group III at 3 months (Left: BMP-2 site, Right: βGal site).

FIG. 13 is a series of 3D CT images. 13A is a 3D CT image of Group IV at 6 weeks (Left: BMP-2 site, Right: βGal site). 13B is a 3D CT image of Group IV at 3 months (Left: BMP-2 site, Right: βGal site).

FIG. 14 is a series of photographs of the H & E staining. 14A is a H & E staining of the experimental site showed cancellous bone formation (200×). 14B is a H & E staining of the control site showed no bone formation (200×).

FIG. 15 is a series of photographs of the Von Kossa staining of the BMP-2 site. 15A depicts lamilar bone formation (50×). 10B depicts higher power field of the bone formation at the BMP-2 site.

FIG. 16 is a series of photographs of the immunochemical stain for detecting adenovirus in Group 4. 16A: positive in pre-implantation culture (200×). 16B: negative in 6 weeks; and 16C: 3 months samples (200×).

### DETAILED DESCRIPTION OF THE PREFERRED EMBODIMENTS

While the present invention may be embodied in many different forms, several specific embodiments are discussed herein with the understanding that the present disclosure is to be considered only as exemplification of the principles of the invention, and it is not intended to limit the invention to the embodiments illustrated.

It will be apparent to those skilled in the art that various modifications and variations can be made in the methods and compositions of the present invention without departing from the spirit or scope of the invention. Thus, it is intended that the present invention include modifications and variations that are within the scope of the appended claims and their equivalents.

The present invention is directed to methods, kits, combinations and pharmaceutical compositions for treating or repairing bone and other tissue defects. Besides being useful for human treatment, the present invention is also useful for veterinary treatment of mammals, exotic animals and farm animals.

The use of the term “about” in the present disclosure means “approximately,” and use of the term “about” indicates that amounts and dosages slightly outside the cited ranges may also be effective and safe, and such amounts and dosages are also encompassed by the scope of the present claims.

The phrase “cell/polymer composition” or “pharmaceutical composition” or simply “formulation” or “composition” refers to the combination of the genetically engineered cells disclosed herein and at least one pharmaceutically acceptable polymer or carrier appropriate for use as a pharmaceutical product.

The phrase “pharmaceutically acceptable” is used adjectivally herein to mean that the modified noun is appropriate for use in a pharmaceutical product. Pharmaceutically acceptable polymers and biomaterials to be used as a matrix or carrier for the therapeutic cells, and for implanting into the defect include, but are not limited to, alginate (Sigma), alginate (Pronova), alginate-RGD, and collagen I (Pancogen). Other pharmaceutically acceptable polymers and biomaterials include any natural or synthetic polymers such as PDS, PLA, PGA, PALGA, PLGA, hydroxyapatite, and the like.

The phrase “bone or other tissue defect” means a defect or injury in or to the bone, cartilage, muscle, adipose or other fibrous tissue.

A “bone or other tissue defect-effective amount,” or “effective amount” is intended to qualify the amount of cell/polymer composition required to treat a bone or other tissue defect in a mammal, or relieve to some extent one or more of the symptoms associated with, or related to, a bone or other tissue defect in a mammal.

The compositions of the present invention are used in a “effective amount.” This means that the concentration of the BMP protein is such that a therapeutic level of protein is delivered over the term that the cell/polymer composition is to be used. Such delivery is dependent on a number of variables including the time period for which the individual dosage unit is to be used, the flux rate of the therapeutic agent, for example, BMP protein, from the cells and polymer, surface area of application site, etc. It is understood, however, that specific dose levels of the therapeutic agents of the present invention for any particular patient depends upon a variety of factors including the activity of the specific compound employed, the age, body weight, general health, sex, and diet of the patient, the time of administration, the rate of excretion, the drug combination, and the severity of the particular disorder being treated and form of administration. Treatment dosages generally may be titrated to optimize safety and efficacy.

The phrase “combination therapy” embraces the administration of BMP producing cells of various types, or employing the inventive formulations with other drugs or therapies. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days, weeks, months or years depending upon the combination selected). “Combination therapy” generally is not intended to encompass the administration of two or more of these therapeutic agents as part of separate monotherapy regimens that incidentally and arbitrarily result in the combinations of the present invention. “Combination therapy” is intended to embrace administration of these therapeutic agents in a sequential manner, that is, where each therapeutic agent is administered at a different time, as well as administration of these therapeutic agents, or at least two of the therapeutic agents, in a substantially simultaneous manner. Sequential or substantially simultaneous administration of each therapeutic agent can be effected by any appropriate route including, but not limited to, topical, oral routes, percutaneous routes, intravenous routes, intramuscular routes, and direct absorption through mucous membrane tissues. The therapeutic agents can be administered by the same route or by different routes. “Combination therapy” also can embrace the administration of the therapeutic agents as described above in further combination with other biologically active ingredients.

Examples of suitable pharmaceutically acceptable formulations are stated herein. Additionally, drug formulations are discussed in, for example, Remington's The Science and Practice of Pharmacy (2000) and Liberman, H. A. and Lachman, L., Eds., Pharmaceutical Dosage Forms, Marcel Decker, New York, N.Y., 1980.

### EXAMPLE 1

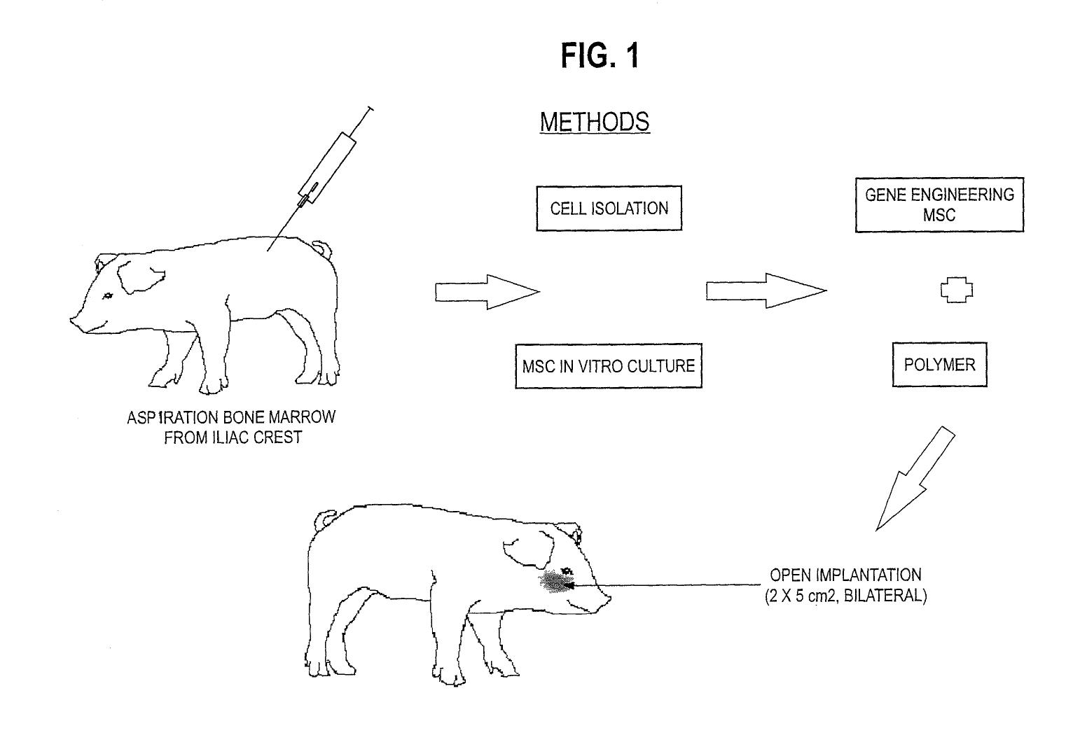

In this example, the repair of critical size craniofacial bone defect in mini-pigs by BMP-2 gene engineered autologous bone marrow stromal cells (MSC) was assessed. The MSC were separated from the aspirate from the iliac crest from 40 minipigs one month before implantation. The general method is illustrated schematically in FIG. 1. As shown below, near-complete critical-size craniofacial defect repair was achieved by simple aspiration of bone marrow stromal cells and after one month implantation of tissue engineered constructs.

### Materials and Methods

Construction of Recombinant Adenovirus

Recombinant adenovirus BMP-2 was constructed by in vivo homologous recombination in 293 cells between expression vector pAC-cytomegalovirus BMP-2 and the large Cla I fragment (2.6-100 mm) of adenovirus type 5 genome DNA. Briefly, to make recombinant plasmid pAC-cytomegalovirus BMP-2, the human BMP-2 gene (No. 40345; ATCC, (American Type Culture Collection), Rockville, Md., U.S.A.) was inserted into the vector pAC cytomegalovirus. The resulting plasmid was made linear by Xho I and cotransfected with the large Cla I fragment of wild-type adenovirus type 5 genome DNA by lipofectamine (GIBCO BRL, Grand Island, N.Y., U.S.A.). For each 35-mm dish of 293 cells, 2 mg of each kind of DNA (total: 4 mg) was transfected. Plaques containing recombinant adenovirus were picked after 10 days of agarose overlay and incubation at 37° C. The viral stocks were propagated and titered in 293 cells. The recombinant adenovirus with human BMP-2 gene, which was under transcriptional control of the cytomegalovirus early gene promoter/enhancer, is known as adenovirus BMP-2 (Lou, J., et al., 1999, Gene Therapy: Adenovirus-Mediated Human Bone Morphogenetic Protein-2 Gene Transfer Induces Mesenchymal Progenitor Cell Proliferation and Differentiation In Vitro and Bone Formation In Vitro, J. ORTHO. RES. 17:43-50). The recombinant adenovirus containing the bacterial β-galactosidase (lac z) gene was constructed by the same procedure (Lou, J., et al. 1996, Adenovirus-Mediated Gene Transfer into Tendon and Tendon Sheath, J. ORTHOP. RES. 14:513-517), and the virus is known as adenovirus β-galactosidase. Viral stock of 293 lysate was titered in 293 cells by serial dilution and plaque-formation assay.

Western Blot Detection of Adenovirus BMP-2 Expression in MSC (Bone Marrow Stromal Cells) In Vitro

Cells from MSC were grown in Dulbecco's modified Eagle medium with 10% fetal calf serum to 90% confluence. The cells were infected with adenovirus b-galactosidase or adenovirus BMP-2 at a multiplicity of infection of 50. The infected cells were maintained in Dulbecco's modified Eagle medium with 10% fetal calf serum and 50 mg/ml ascorbic acid. The medium was collected 7 days later and then immunoprecipitated with monoclonal antibody h4b2/5.10.24 (a gift from Genetics Institute, Cambridge, Mass., U.S.A.). The immunoprecipitation was then run on a 12.5% sodium dodecyl phosphate-polyacrylamide gel electrophoresis and transferred overnight to a polyvinylidene difluoride membrane (Millipore, Bedford, Mass., U.S.A.). The expressed protein was detected with monoclonal antibody h4b2/5.10.24 and goat anti-mouse IgG alkaline-phosphatase conjugate (Sigma Chemical, St. Louis, Mo., U.S.A.).

Bone Marrow Aspiration and Adenoviral Infection

40 minipigs (Mitsae Pig, CGU, Taiwan) were randomly assigned to four groups. The four groups (n=10) of different polymers: Group I, Alginate (Pronova); Group II, Alginate (Sigma: self-purified); Group III, Alginate (RGD) and Group IV Pancogene S (Gattefosse). The minipigs were sacrificed at 6 weeks and at 3 months.

At the first stage of surgery, bone marrow aspiration was performed when minipigs were under intramuscular injection of 1 ml/10 kg 2% Rompum (Bayer, USA), Ketamine injection (50mg/ml, Yung Shin Pharmaceutical Industrial Co., Taiwan). 20 cc of the aspirated bone marrow was mixed with 2 cc of heparin sodium (5000 unit/ml, Agglutex, China Chemical & Pharmaceutical Co., Taiwan). Aspirated iliac crest marrow (5-10 ml) was transferred to sterile tubes, to which 20 ml complete medium was added. The tubes were spun at 1000 rpm for 5 min to pelletize the cells, the supernatants and fat layers were removed, and the cell pellets (2.5-5.0 ml) were resuspended and loaded onto 70% Percoll (Sigma, St. Louis, Mo.) gradients. These gradients were centrifuged at 460× g for 15 min and harvested into three fractions with a pipet: the top 25% of the gradients (low density cells), pooled density=1.03 g per ml; the middle 50% of the gradients (high density cells), pooled density=1.10 g per ml; density=1.14 g per ml. In preliminary experiments, each of these three pools was plated separately in complete medium in 100 mm dishes. Adherent marrow-derived mesenchymal cells were observed to be localized to the low-density fraction. To produce adherent cell cultures for all subsequent experiments, only the cells in the low density fraction were plated. The medium was changed every 3-4 days. After one month, an adequate number of cells was reached.

At the experimental site, adenovirus mediated human BMP-2 gene transfer to MSCs was performed 5-7 days before the secondary stage of surgery. As a control, adenovirus mediated βGal gene transfer was carried out at the same time and for the same period of time. The cells were trypsinized by 25% trypsin and washed for three times. Cell numbers were determined with a hemocytometer. The cells were then mixed with four different polymers to achieve the same concentration of 50×10⁶/ml. After one hour in the incubator, the cells in group 4 with the polymer Pancogene S became more gel-like (in the dishes). In groups 1, 2 and 3, the cells in CaSO₄/alginate were gels ten minutes after mixing.

Open Implantation on the Cranial Defects After Creating Cranial Bony Defect

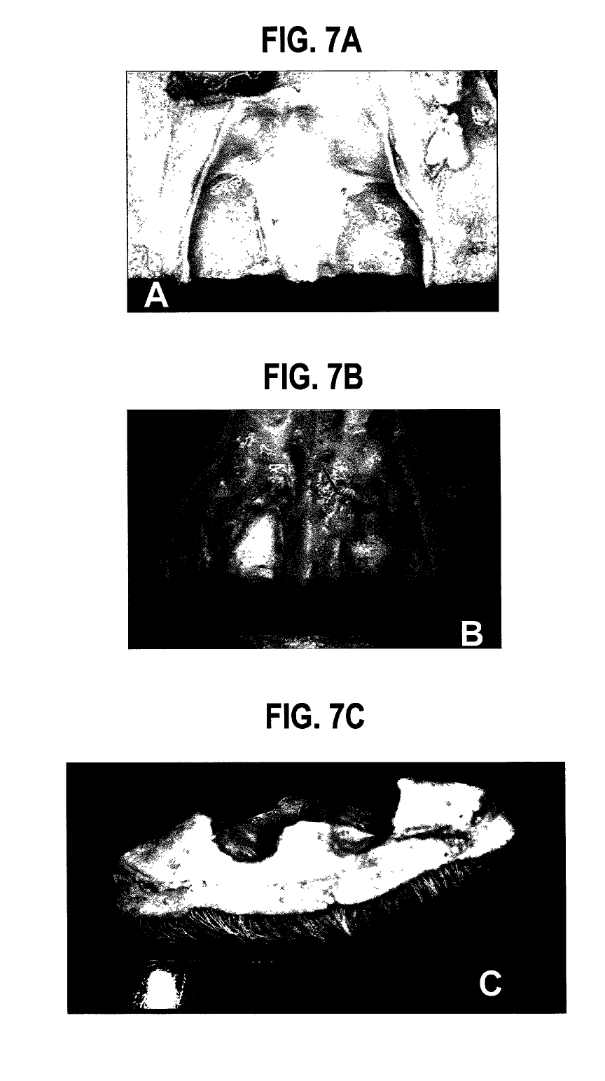

At the secondary stage of surgery, all minipigs were intubated and on ventilators. Two cranial defects of 2×5 cm² each were created by removal of periosteum from above and dura from below, ensuring that no osteoinductive tissue existed (FIG. 7). The dura defects were replaced with acellular dermis (Pig Alloderm, LifeCell USA).

3ml of cell/polymer constructs were implanted to each site. The right sided defect was covered with adenovirus-mediated BMP-2 gene transfered MSCs/polymer. The left side defect was filled with adenovirus-mediated βGal gene transfered on MSCs/polymer. The scalp wounds were closed in a watertight manner.

Histologic Examination of Bone—Samples Harvested from the Cranial Defects

Harvested samples were fixed in buffered 10% formalin for 72 hours and then sawed in half. Half of them were decalcified in Decalcifier I (Surgipath, Northbrook, Ill., U.S.A.) solution for another 48 hours. The samples then underwent procedures for embedding, incising, and staining with hematoxylin and eosin. The nondecalcified other half of the samples was stained with von Kossa's silver nitrate for demonstration of matrix mineralization (Heise, C., et al., 1997).

Immunohistochemistry for Adenovirus Hexon Protein

Immunohistochemistry was performed using formalin-fixed, paraffin embedded tumor sections. After removal from paraffin, slides were hydrated and digested with pronase (Meridian Diagnostics, Cincinnati, Ohio). The primary antibody (MAB805, Chemicon International, Temecule, Calif.) was applied at 1:500 dilution, and slides were incubated for 1 hr at 35° C. A biotinylated goat anti-mouse secondary was then applied, followed by a streptavidin-horseradish peroxidase conjugate. Diaminobenzidine (DAB) was used as the chemogen before slides were counterstained with hematoxylin.

Biomechanical Analysis

The samples were stored at −70° C. before testing. All samples of engineered bones were sawed using a 6-mm dermal punch to form disks. Each disk was immediately mounted in an electrically insulating cylindrical confining chamber. The chamber was mounted in a servo-controlled Dynastat mechanical spectrometer (IMASS, Hingham, Mass.) interfaced to a computer (Lou, J., et al., 1996, Adenovirus-Mediated Gene Transfer into Tendon and Tendon Sheath, J. ORTHOP. RES. 14:513-517). The samples were equilibrated at room temperature in 0.15M PBS, pH 7.4, containing 100 units/ml penicillin G, and 100 ug/ml streptomycin. Samples were compressed between a porous polyethylene platen at the base of the chamber.

After mounting each disk in the confined compression chamber, the distance between the porous platen and the chamber was decreased until a signal of ˜5 gm was detected by the load cell. This distance was taken to be the sample thickness. Each disk was compressed by 10 sequential increments of 2.5-3.0% static strain, up to a maximum of 25-30% total strain. After each increment, the load was recorded every 0.5 seconds for 100 seconds. Stress relaxation data was fit to a poroelastic model of material behavior that yielded values for the material properties; equilibrium modulus.

Statistical Analysis

All measurements were collected in triplicate and expressed as means ±standard deviations. Single factor analysis of variance (ANOVA) was employed to assess the statistical significance of results for all the 3D CT images of the repaired bone areas.

### Results

Recombinant Adenovirus Construction and Human BMP-2 Expression

Adenovirus BMP-2, a replication-defective adenovirus vector encoding human BMP-2, was constructed in applicants laboratory. In this vector, transcription of the BMP gene is controlled by cytomegalovirus early gene promoter/enhancer. The recombinant adenovirus encoding Escherichia coli lac z gene (adenovirus bgalactosidase) was constructed by the same procedure as the control vector. These two recombinant adenoviruses have been deleted only in the E1 region of the genome. The E3 region of the virus genome was purposefully retained intact on the vectors because of evidence that the E3 proteins of the virus inhibit the host immune response (Horwitz, M. S., et al., 1995, Model System for Studying the Effects of Adenovirus E3 Gene on Virulence in Vivo, CURR. TOPICS MICROBIOL. IMMUNOL. 199:195-211). Titered in 293 cells, the viral lysate of the vectors are usually 10⁸ plaque-forming units per milliliter.

The gene expression of human BMP-2 protein in MSCs, which was mediated by adenovirus BMP-2 gene transfer, was assessed by detecting the coding product with monoclonal antibody h4b2/5.10.24 in transduced cells. The secreted active BMP-2 molecules were detected in cultured medium from adenovirus BMP-2-transduced MSCs by immunoprecipitation and immunoblot analysis but were not found in cultured medium from either adenovirus b-galactosidase-infected MSCs. The BMP gene transfected by adenovirus seems well expressed and processed in MSCs. The molecular weight of secreted BMP-2 indicated mature status without prepeptide. The band was noticeable at 18 KDa due to overlap of bands at 16 and 22.

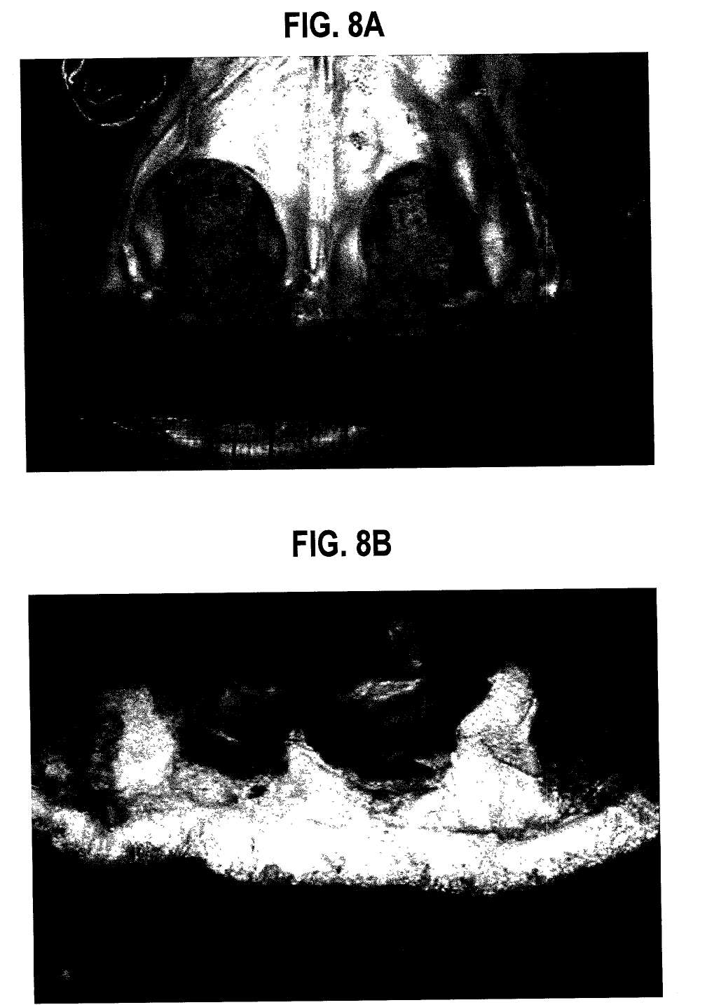

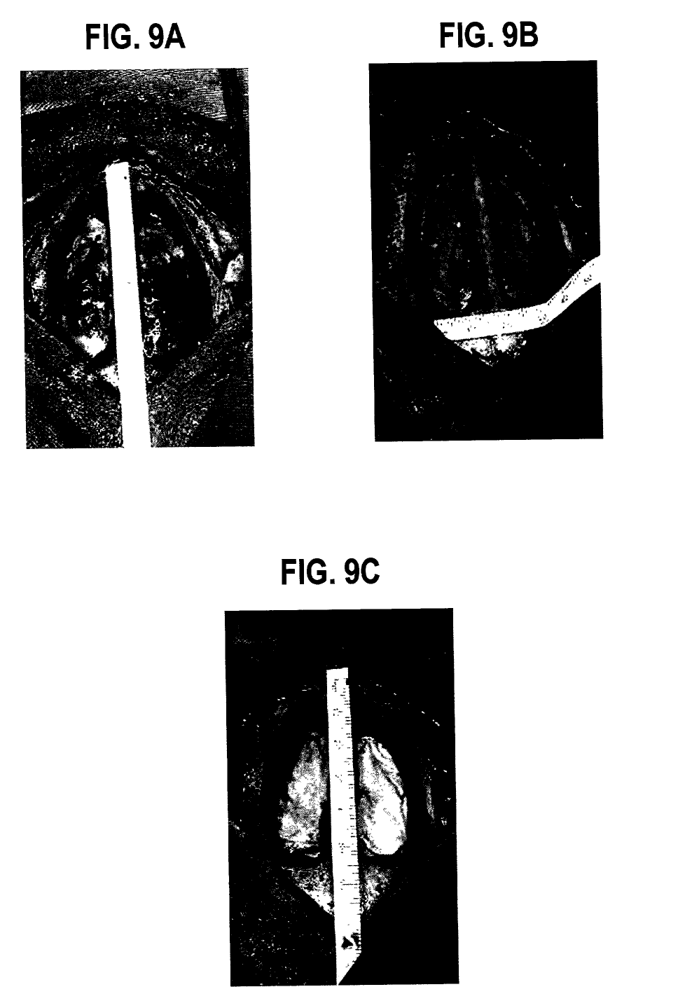

Grossly, in group 1, 2, 3, there is no bone formation at cranial defects sites. In Group 1, after 3 months of in vivo treatment, incomplete repair was detected (FIG. 8). There was noticeable whitish bone formation at the site infected with BMP-2 from 6 weeks on (FIG. 9A) while no obvious bone formation was found at the βGal infection site (FIG. 9).

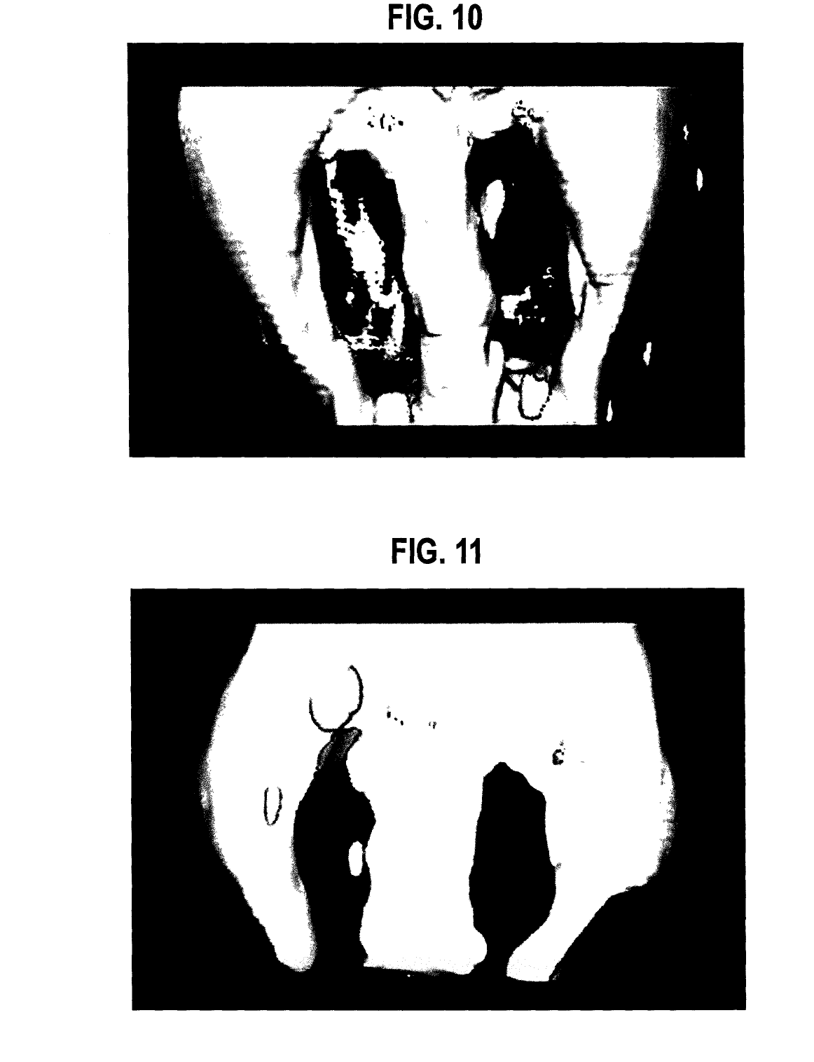

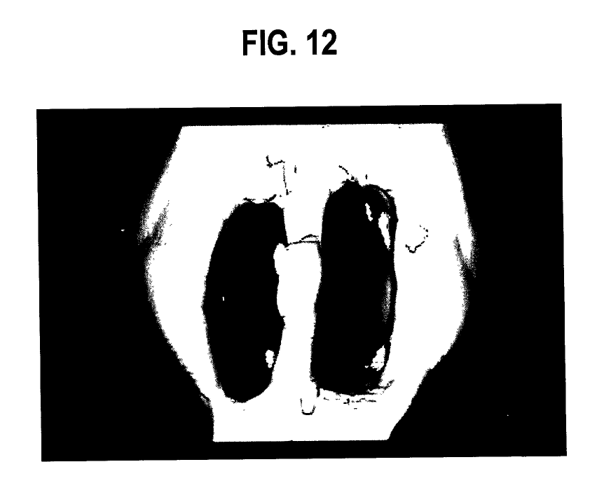

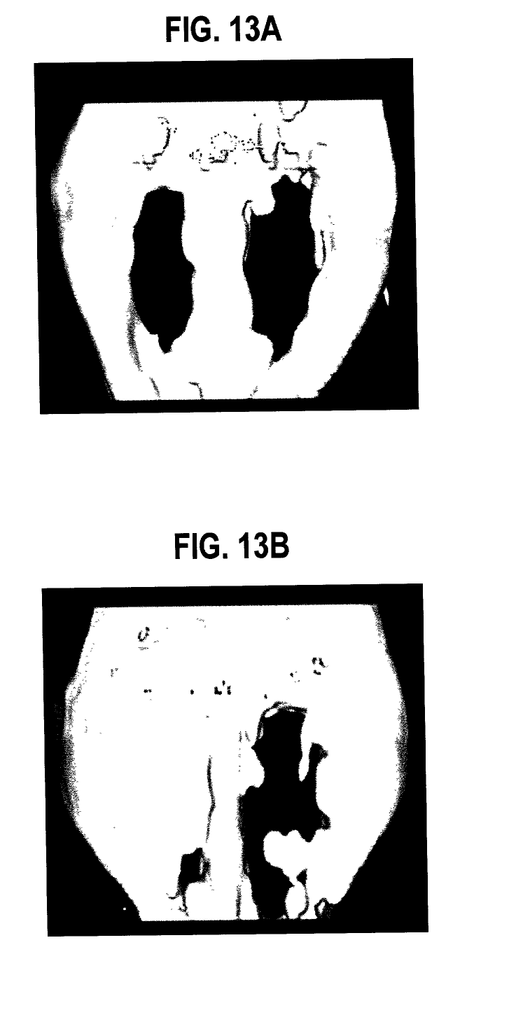

3D-CT image studies were performed for each sample. In group I, there were some scattered bony formations at both reconstructed areas (FIG. 10). In group II, there was little bone regeneration in the defect sites (FIG. 11). In Group III, hardly any bony regeneration was noted (FIG. 12). The 3D CT image demonstrated some bony formation in Group IV at 6 weeks (FIG. 13A), and near-complete repair of the defective area at 3 months (FIG. 13B).

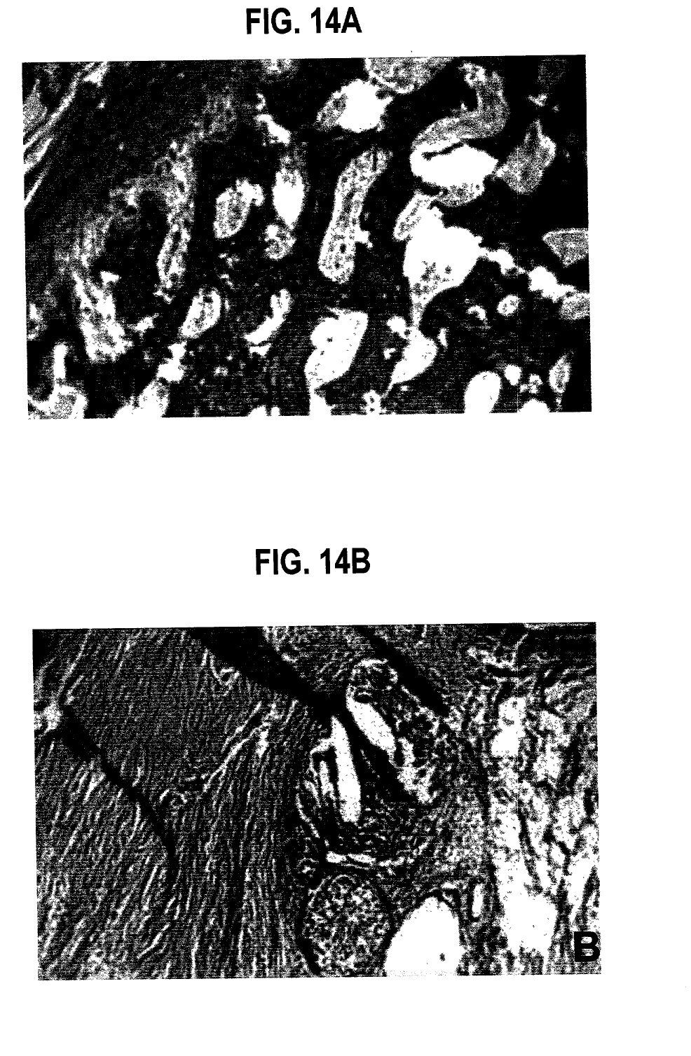

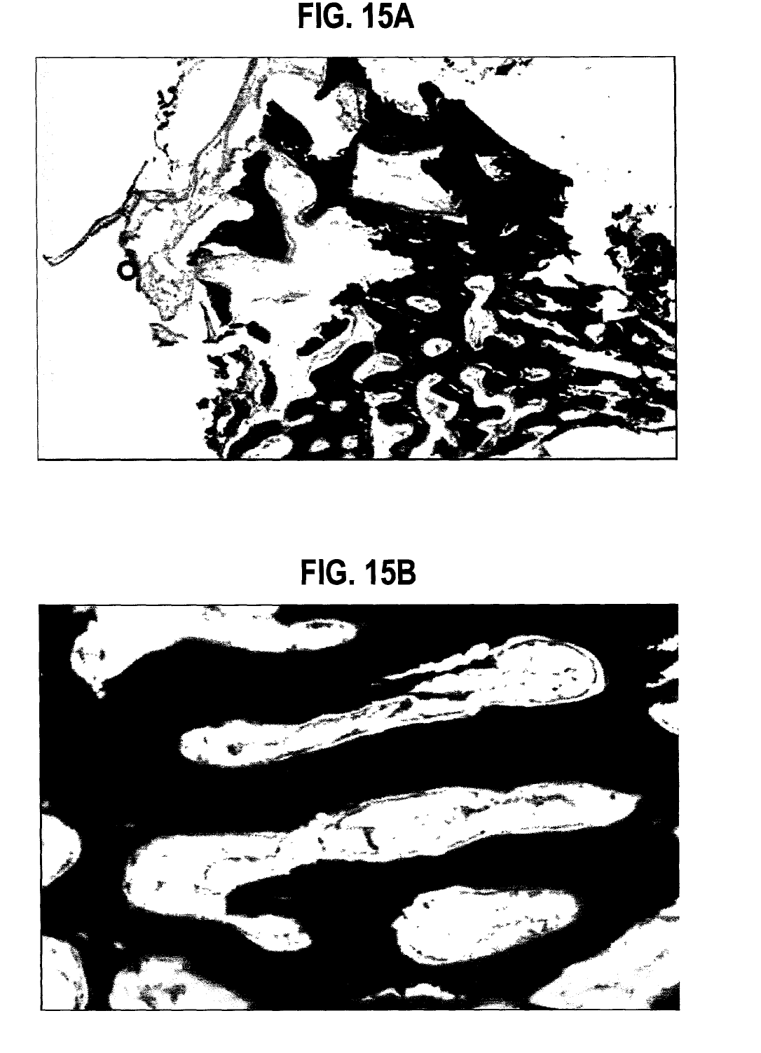

The H & E staining of Groups 1, 2 and 3 did not demonstrate good bone formation. In Group IV, the experimental site showed cancellous bone formation (FIG. 14A), while the control site did not (FIG. 14B). The Von Kossa stains of the experimental site were strongly positive for mineralization, and no cortical formation at 3 months in vivo (FIG. 15).

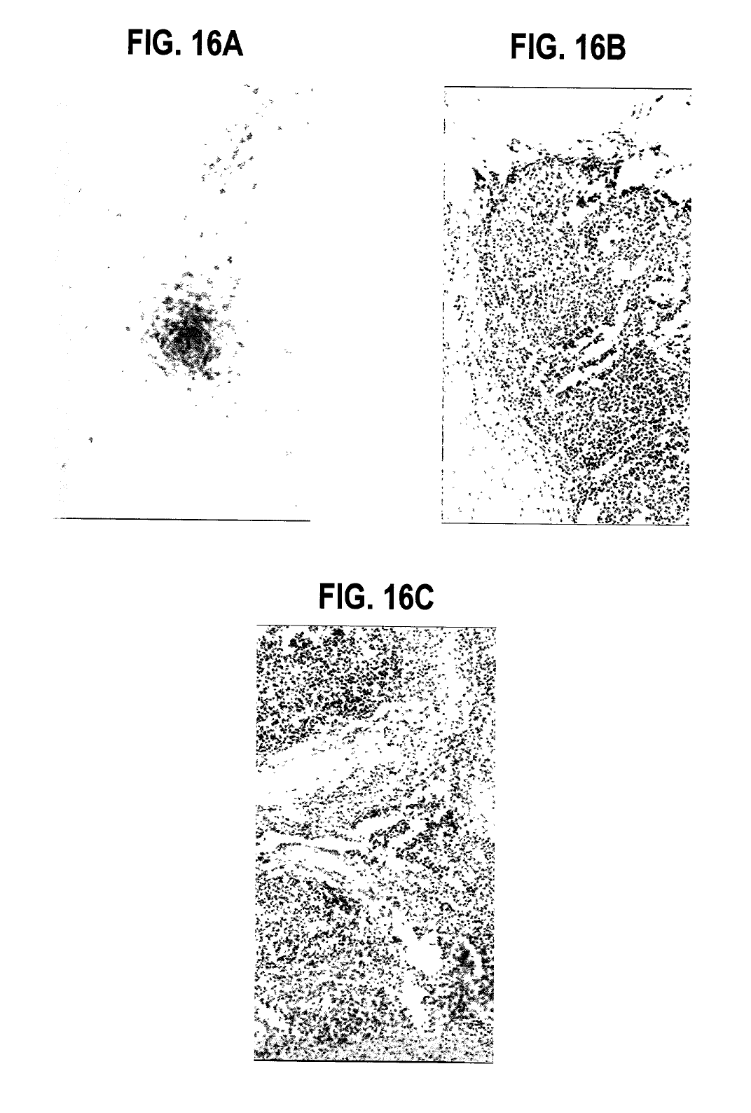

The immunochemical stain for detecting adenovirus was positive in all pre-implantation culture (FIG. 16A) and was negative in all 6 weeks and 3 months samples (FIGS. 16B, 16C).

At 6 weeks, there were no statistically significant differences between the four groups. At 3 months, in Group IV, there was a significant increase of bone formation of the defective site induced by adenovirus bone morphogenetic protein (BMP)-2 transduction.

Table 1 shows the new bone formation areas by 3D CT (cm ²). The statistical data revealed that Group IV, at 3 months, had significant increase of bone formation of the defective site induced by adenovirus bone morphogenetic protein (BMP)-2 transduction.

TABLE 1
New Bone Formation Areas by 3D CT (cm²)

| Group | | | |
|---|---|---|---|
| (n = 5)/ | | | |
| area, cm2 | Experimental | Control | |
| 1 (6w) | 3.31 | 2.13 | 0.3789 |
| 1 (3M) | 4.51 | 2.87 | 0.2141 |
| 2 (6w) | 3.17 | 2.35 | 0.2664 |
| 2 (3M) | 4.17 | 3.97 | ns |
| 3 (6w) | 1.37 | 1.01 | ns |
| 3 (3M) | 4.17 | 2.72 | ns |
| 4 (6w) | 4.32 | 3.61 | ns |
| 4 (3M) | 6.32 | 4.41 | p = 0.0128 |

The constructed recombinant adenovirus with human BMP-2 gene successfully mediated the gene transfer and BMP-2 protein expression in the MSCs and produced an effective amount of the protein. Expressed BMP-2 protein was detected in culture medium of the transduced cell. The proper size of the detected protein suggested that adenovirus-mediated BMP-2 gene transfer and expression underwent the proper process in protein secretion and enzymolysis after translation. One band of BMP-2 protein has been observed in a Western blot analysis, and a similar pattern has also been found in a Chinese hamster ovary (CHO) cell (Cook, S. D., et al., 1994, In Vivo Evaluation of Recombinant Human Osteogenic Protein Implants as a Bone Graft Substitute for Spinal Fusions, SPINE 19:1655-1663) and osteosarcoma (Wang, E. A., et al., 1990, Recombinant Human Bone Morphogenetic Protein Induces Bone Formation, PROC NATL ACAD SCI USA 87:2220-2224) expression media. This pattern may result from unclear proteinic enzymolysis.

Adenovirus-mediated BMP-2 gene transfer on MSCs directed the cells to proliferation and differentiation. Cell response to the gene transfer may have been through an autocrine or paracrine effect of BMP-2 protein that was expressed from the transferred BMP-2 gene by the cell itself.

The results demonstrated that bone repair in vivo was induced and enhanced by adenovirus bone morphogenetic protein (BMP)-2 transduction. Moreover, gene transfer has the advantage of directing the cell to continuously produce BMP-2 protein. In an in vitro study, a one-time transduction of recombinant adenovirus into the cells kept the cell exposure to BMP-2 until mineralization; this required continued incubation for as long as 30 days and several medium changes (Lou, J., et al., 1999, Gene Therapy: Adenovirus-Mediated Human Bone Morphogenetic Protein-2 Gene Transfer Induces Mesenchymal Progenitor Cell Proliferation and Differentiation In Vitro and Bone Formation In Vitro, J. ORTHO. RES. 17:43-50). BMP-2 protein should continue to be released as long as the transduced gene stays in the cell.

Recombinant adenovirus-mediated gene transfer has its limitation in the immune-competent host. The host immune response to the adenovirus proteins from leak expression of virus genome will delete the recombinant adenovirus and terminate expression of the transgene. Also, inflammation has been observed. Therefore, the use of a new generation of recombinant adenovirus vectors that has less immunogenecity and longer expression in the immune-competent host will help to reduce these problems and thus be more effective.

This study demonstrated that BMP-2 gene transfer into mesenchymal stem cells could potentially direct differentiation into bone formation in vivo. Furthermore, this study demonstrated that the use of the polymer Pancogene S/MSCs with adv-BMP-2 gene transfer will enhance the bony healing of critical size craniofacial defect. Table 2, below, shows the biomechanic test in Group IV experimental site compared with the normal cranium bone.

TABLE 2
Biomechanics test (MPa)

| | | |
|---|---|---|
| normal cranial bone | 88646.02 ± 5121.803 | P = 0.3444 |
| Group 4 (collagen) | 80536.46 ± 19302.64 | |

### EXAMPLE 2

In a second example, the bone marrow stromal cells (MSC) were separated from the aspirate from the iliac crest from 20 minipigs one month before implantation. The MSC were expanded in monolayered culture. Full-thickness bone defects (3×1.2 cm²) were created on the bilateral maxilla of the minipigs. The osteogenic periosteum was removed. The experimental site (on the animal's left), the MSC was implanted by using ex vivo adenovirus-BMP-2 (Bone Morphogenic Protein-2) mediated gene transfer to the expanded MSC 5 days before implantation with a concentration was 5×10⁷/ml of collagen type I (Pancogen). In the control site (on the animal's right), the MSC was implanted by ex vivo adenovirus-βGal medicated gene transfer under the same condition. After 3 months, 20 minipigs were sacrificed, the head sent for 3D CT examination and then sawed in half. One half was preserved in −80° C. refrigerator for further biomechanic testing. The other half was sawed into slices for histology (HE and Von Kossa stain) and immunochemical stain for detection of adenovirus.

Near-complete maxilla defect repair was achieved by simple aspiration of bone marrow stromal cells and after three months implantation of tissue engineered constructs with collagen type I. The histology showed mature woven bone formation. The regenerated bone and the biomechanic test are similar to that of normal cranial bone. This is a cell therapy which is further based on ex vivo infection, thus the gene is securely delivered to target cells and the viral contact to the host animal will be significantly reduced.

### Materials And Methods

Construction of Recombinant Adenovirus

Recombinant adenovirus BMP-2 was constructed by in vivo homologous recombination in 293 cells between expression vector pAC-cytomegalovirus BMP-2 and the large Cla I fragment (2.6-100 mm) of adenovirus type 5 genome DNA. Briefly, to make recombinant plasmid pAC-cytomegalovirus BMP-2, the human BMP-2 gene (no. 40345; ATCC [American Type Culture Collection], Rockville, Md., U.S.A.) was inserted into the vector pAC cytomegalovirus. The resulting plasmid was made linear by Xho I and cotransfected with the large Cla I fragment of wild-type adenovirus type 5 genome DNA by lipofectamine (GIBCO BRL, Grand Island, N.Y., U.S.A.). For each 35-mm dish of 293 cells, 2 mg of each kind of DNA (total: 4 mg) was transfected. Plaques containing recombinant adenovirus were picked after 10 days of agarose overlay and incubation at 37° C. The viral stocks were propagated and titered in 293 cells. The recombinant adenovirus with human BMP-2 gene, which was under transcriptional control of the cytomegalovirus early gene promoter/enhancer, was named adenovirus BMP-2. The recombinant adenovirus containing the bacterial β-galactosidase (lac z) gene was constructed by the same procedure, and the virus was named adenovirus β-galactosidase. Viral stock of 293 lysate was titered in 293 cells by serial dilution and plaque-formation assay.

Western Blot Detection of Adenovirus BMP-2 Expression in MSC (Bone Marrow Stromal Cells) In Vitro

Cells from MSC (Bone Marrow Stromal Cells) were grown in Dulbecco's modified Eagle medium with 10% fetal calf serum to 90% confluence. The cells were infected with adenovirus β-galactosidase or adenovirus BMP-2 at a multiplicity of infection of 50. The infected cells were maintained in Dulbecco's modified Eagle medium with 10% fetal calf serum and 50 mg/ml ascorbic acid. The medium was collected 7 days later and then immunoprecipitated with monoclonal anti-body h4b215.10.24 (a gift from Genetics Institute, Cambridge, Mass., U.S.A.) The immunoprecipitation was then run on a 12.5% sodium dodecyl phosphate-polyacrylamide gel electrophoresis and transferred overnight to a polyvinylidene difluoride membrane (Millipore, Bedford, Mass., U.S.A.). The expressed protein was detected with monoclonal antibody h4b2/5.10.24 and goat anti-mouse IgG alkaline-phosphatase conjugate (Sigma Chemical, St. Louis, Mo., U.S.A.).

Bone Marrow Aspiration and Adenoviral Infection

20 female minipigs (Mitsae Pig, CGU, Taiwan) were used in this experiment. The minipigs were sacrificed at 3 months after tissue enginereered constructs implantation. At the first stage of surgery, bone marrow aspiration was performed when minipigs were under intramuscular injection of 1 ml/10 kg 2% Rompum (Bayer, USA), Ketamine injection (50 mg/ml, Yung Shin Pharmaceutical Industrial Co., Taiwan). 20 cc of the aspirated bone marrow was mixed with 2 cc of heparin Sodium (5000 unit/ml, Agglutex, China Chemical & Pharmaceutical Co., Taiwan). Aspirated iliac crest marrow (5-10 ml) was transferred to sterile tubes, to which 20 ml complete medium was added. The tubes were spun at 1000 rpm for 5 min to pellet the cells, the supernatants and fat layers were removed, and the cell pellets (2.5-5.0 ml) were resuspended and loaded onto 70% Percoll (Sigma, St. Louis, Mo.) gradients. These gradients were centrifuged at 460× g for 15 min and harvested into three fractions with a pipet: the top 25% of the gradients (low density cells), pooled density=1.03 g per ml; the middle 50% of the gradients (high density cells), pooled density=1.10 g per ml; density=1.14 g per ml. In preliminary experiments, each of these three pools was plated separately in complete medium in 100 mm dishes. Adherent marrow-derived mesenchymal cells were observed to be localized to the low-density fraction. To produce adherent cell cultures for all subsequent experiments, only the cells in the low density fraction were plated. The medium was changed every 3-4 days. After one month, an adequate number of cells was reached (FIG. 1).

At the experimental site, Adenovirus mediated human BMP-2 gene transfer to MSCs was performed 5 days before the secondary stage of surgery (FIG. 1). As a control, Adenovirus mediated βGal gene transfer was carried out at the same time and for the same period of time. The cells were trypsinized by 25% trypsin and washed for three times. Cell numbers were determined with a hemocytometer. The cells were then mixed with four different polymers to achieve the same concentration of 50×10⁶/ml. After one hour in the incubator, the cells with the polymer Pancogene S became more gel-like.

Open Implantation on the Maxilla Defects After Creating Maxillary Bone Defect

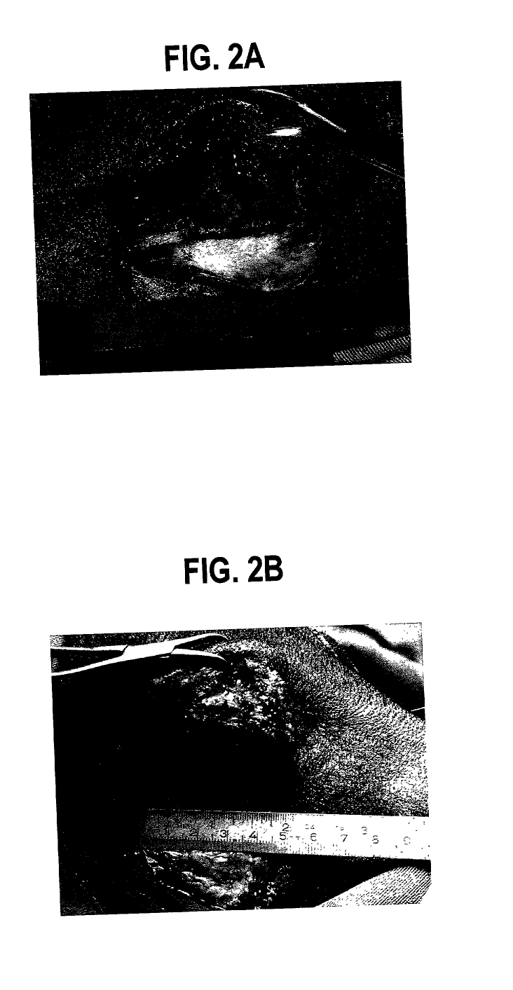

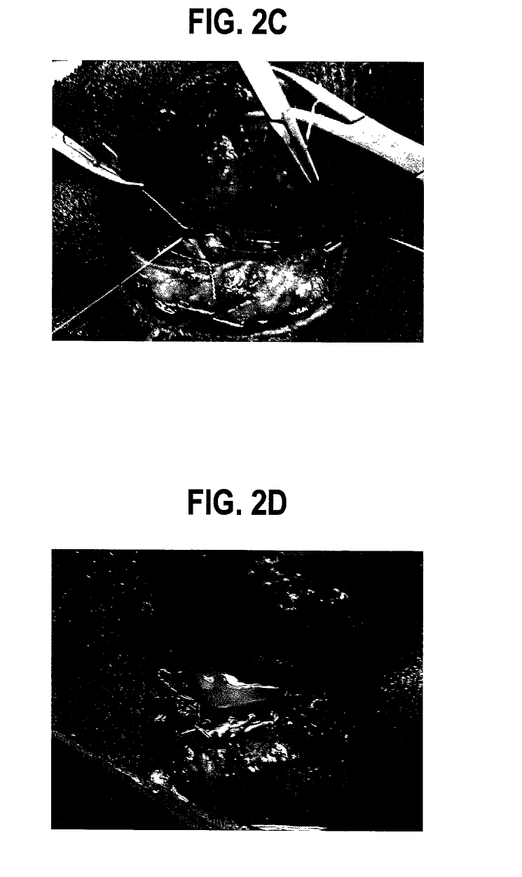

At the secondary stage of surgery, all minipigs were intubated and on ventilators. Bilateral maxillary defects of 3×1.2 cm² each were created by removal of bone and periosteum, confirming that no osteoinductive tissue exists (FIG. 2 A, B). To bridge the defect, one biodegradeable PLLA 6-holes miniplate was used on each maxillary defect (FIG. 2C). Each 3ml of cell/polymer construct was implanted to each site (FIG. 2D). The right sided defect was covered with adenovirus-mediated BMP-2 gene transfered MSCs/polymer. The left side defect was filled with adenovirus-mediated βGal gene transfered on MSCs/polymer. The facial wounds were closed in a watertight manner.

Histologic Examination of Bone Samples Harvested from the Maxilla Defects

Harvested samples were fixed in buffered 10% formalin for 72 hours and then sawed in half. Half of them were decalcified in Decalcifier I (Surgipath, Northbrook, Ill., U.S.A.) solution for another 48 hours. The samples then underwent procedures for embedding, incising, and staining with hematoxylin and eosin. The nondecalcified other half of the samples was stained with von Kossa's silver nitrate for demonstration of matrix mineralization.

Immunohistochemistry for Adenovirus Hexon Protein

Immunohistochemistry was performed using formalin-fixed, paraffin embedded tumor sections. After removal from paraffin, slides were hydrated and digested with pronase (Meridian Diagnostics, Cincinnati, Ohio). The primary antibody (MAB805, Chemicon International, Temecule, Calif.) was applied at 1:500 dilution, and slides were incubated for I hr at 35° C. A biotinylated goat anti-rabbit secondary was then applied, followed by a streptavidin-horseradish peroxidase conjugate. Diaminobenzidine (DAB) was used as the chemogen, before slides were counterstained with hematoxylin.

Biomechanical Analysis

The samples were stored at −70° C. before testing. All the samples of engineered bones were sawed using a 6-mm dermal punch to form disks. Each disk was immediately mounted in an electrically insulating cylindrical confining chamber. The chamber was mounted in a servo-controlled Dynastat mechanical spectrometer (IMASS, Hingham, Mass.) interfaced to a computer. The samples were equilibrated at room temperature in 0.15M PBS, pH 7.4, containing 100 units/ml penicillin G and 100 ug/ml streptomycin. Samples were compressed between a porous polyethylene platen and at the base of the chamber.

After mounting each disk in the confined compression chamber, the distance between the porous platen and the chamber was decreased until a signal of ˜5 gm was detected by the load cell. This distance was taken to be the sample thickness. Each disk was compressed by 10 sequential increments of 2.5-3.0% static strain, up to a maximum of 25-30% total strain. After each increment, the load was recorded every 0.5 seconds for 100 seconds. Stress relaxation data was fit to a poroelastic model of material behavior that yielded values for the material properties; equilibrium modulus.

Statistical Analysis All measurements were collected in triplicate and expressed as means ± standard deviations. Single factor analysis of variance (ANOVA) was employed to assess the statistical significance of results for all the 3D CT images of the repaired bone areas.

### Results

Recombinant Adenovirus Construction and Human BMP-2 Expression

Adenovirus BMP-2, a replication-defective adenovirus vector encoding human BMP-2, was constructed. In this vector, transcription of the BMP gene is controlled by cytomegalovirus early gene promoter/enhancer. The recombinant adenovirus encoding Escherichia coli lac z gene (adenovirus β-galactosidase) was constructed by the same procedure as the control vector. These two recombinant adenoviruses have been deleted only in the E1 region of the genome. The E3 region of the virus genome was purposefully retained intact on the vectors because of evidence that the E3 proteins of the virus inhibit the host immune response. Titered in 293 cells, the viral lysate of the vectors are usually 10⁸ plaque-forming units per milliliter.

The gene expression of human BMP-2 protein in MSCs, which was mediated by adenovirus BMP-2 gene transfer, was assessed by detecting the coding product with monoclonal antibody h4b2/5.10.24 in transduced cells. The secreted active BMP-2 molecules were detected in cultured medium from adenovirus BMP-2-transduced MSCs by immunoprecipitation and immunoblot analysis but were not found in cultured medium from either adenovirus b-galactosidase-infected MSCs. The BMP gene transfected by adenovirus seems well expressed and processed in MSCs. The molecular weight of secreted BMP-2 indicated mature status without prepeptide. The band was noticeable at 18 KDa due to overlap of bands at 16 and 22 KDa.

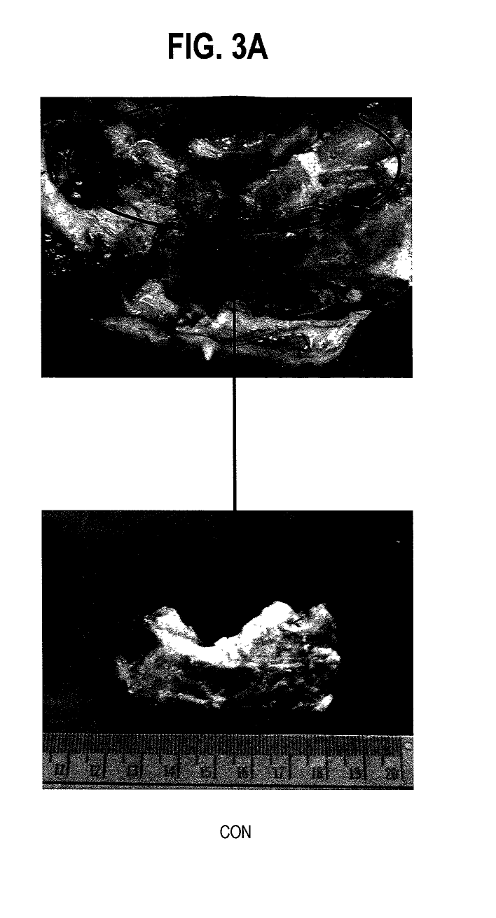

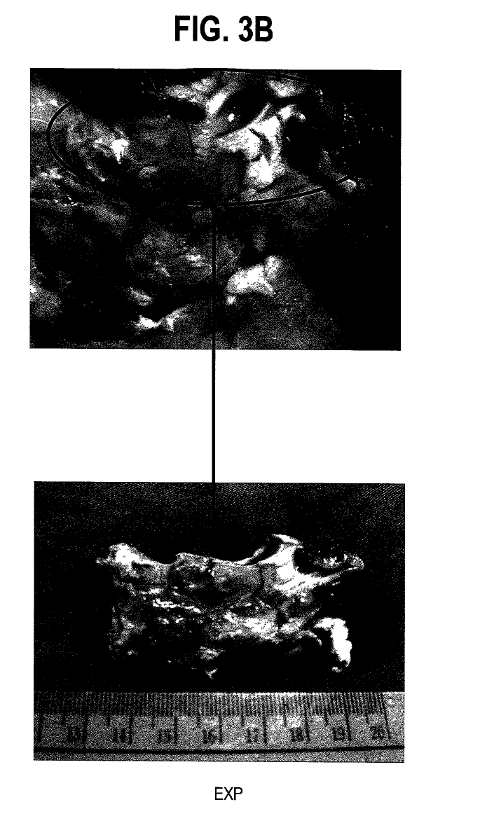

There was noticeable whitish bone formation at the site infected with BMP-2 at 3 months, while no obvious bone formation was found at the βGal infection site (FIG. 3).

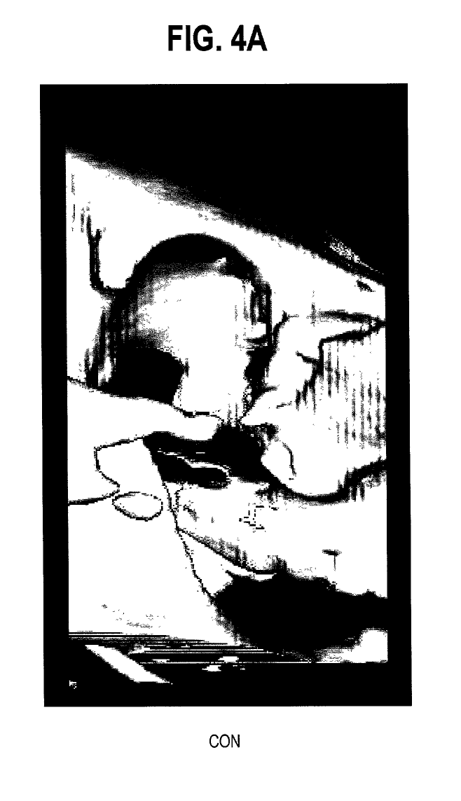

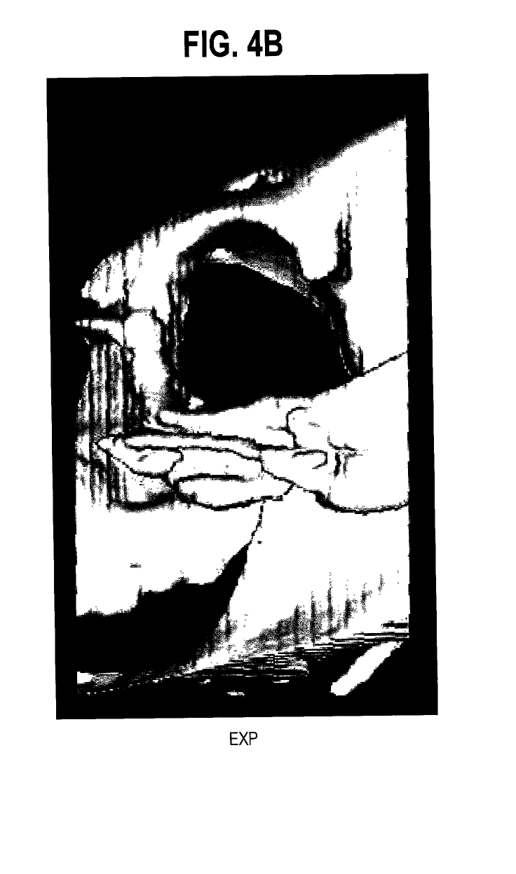

3D-CT image studies were performed for each sample. The 3D CT image demonstrated complete repair of the defect area at 3 month at the experimental site (FIG. 4).

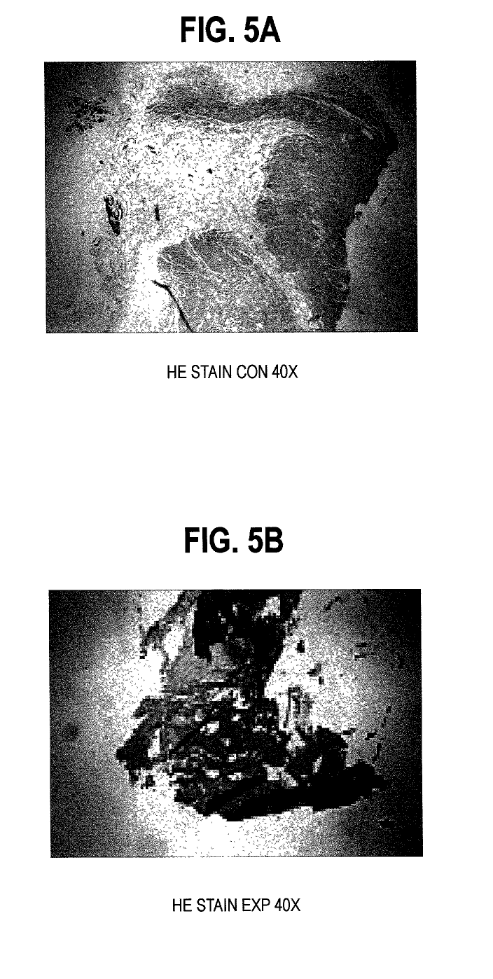

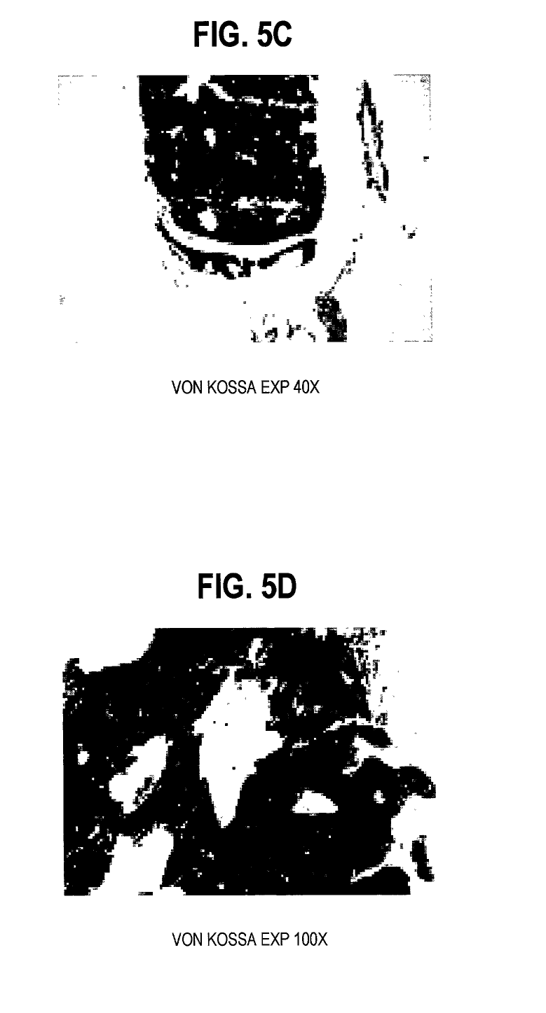

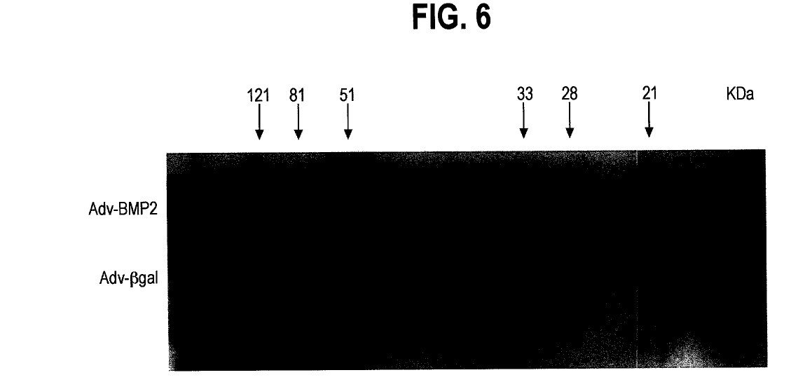

The H & E staining of the experimental site showed cancellous bone formation, while the control site did not (FIG. 5). The Von Kossa stain of the experimental site were strongly positive for mineralization, and mature woven de novo bone formation at 3 months in vivo (FIG. 5).

At 3 months, there was a significant increase of bone formation of the defect site induced by adenovirus bone morphogenetic protein (BMP)-2 transduction as shown in Table 3, below.

TABLE 3
New Bone Formation Areas by 3D CT (n = 10) (cm²)

| | Mean +/− SD |
|---|---|
| Experimental | 2.6275 +/− 0.1795 |
| Control | 1.78 +/− 0.0764 |

p-value = 0.0154

The biomechanical properties between the harvest in the experimental site and normal maxilla bone did not show statistical difference.

The contents of all cited references throughout this application are hereby expressly incorporated by reference. The practice of the present invention will employ, unless otherwise indicated, conventional techniques of pharmacology and pharmaceutics, which are within the skill of the art.

Although the invention has been described with respect to specific embodiments and examples, it should be appreciated that other embodiments utilizing the concept of the present invention are possible without departing from the scope of the invention. The present invention is defined by the claimed elements, and any and all modifications, variations, or equivalents that fall within the true spirit and scope of the underlying principles.

I claim:

1. A pharmaceutical composition, comprising a plurality of bone marrow stromal cells (MSCs) comprising an adenovirus mediated human BMP-2 gene, and a pharmaceutically acceptable polymer.

2. The composition as recited in claim 1 wherein the polymer is selected from a group consisting of alginate and collagen.

3. The composition as recited in claim 1 wherein the MSCs are present in a concentration of about 50×10⁶ per ml of the polymer.

4. The composition as recited in claim 1 wherein the polymer is Pancogene S.

5. A method of treating a bone or other tissue defect, comprising:
a. Obtaining a plurality of MSCs from a subject;
b. transferring a BMP-2 gene to the MSCs to form BMP-2 protein producing MSCs; and
c. implanting the protein producing MSCs to a site on the subject.

6. The method as recited in claim 5 wherein the BMP-2 gene is transferred via an adenovirus.

7. The method as recited in claim 5 further comprising mixing the BMP-2 producing MSCs with a polymer either before, during or after the implantation of the protein producing MSCs.

8. The method as recited in claim 5 wherein the protein producing MSCs implanted are present in a concentration of about 50×10⁶ per ml of a pharmaceutically acceptable polymer and produce an effective amount of the protein.

9. A BMP-2 protein at a site of bone or other tissue defect produced by the method of obtaining a plurality of MSCs from a subject, transferring a BMP-2 gene to the MSCs to form BMP-2 protein producing MSCs, and implanting the protein producing MSCs to the site on the subject.

10. The protein as recited in claim 9 further comprising mixing the BMP-2 producing MSCs with a polymer either before, during or after the time of implantation of the protein producing MSCs.
